(19) United States
(12) Patent Application Publication
(10) Pub. No.: US 2002/0010141 A1
(43) Pub. Date: Jan. 24, 2002

(54) Isoflavones for treatment of obesity

(76) Inventor: Jonathan Ingram, Prides Crossing, MA (US)

Correspondence Address: MCDERMOTT, WILL & EMERY, 600 13th Street, N.W., Washington, DC, 20005-3096, US

(21) Appl. No.: 09/821,750

(22) Filed: Mar. 30, 2001

Related U.S. Application Data
(60) Provisional application No. 60/193,463, filed on Mar. 31, 2000.

(51) Int. Cl. A61K 31/7048; A61K 31/353

(52) U.S. Cl. 514/27; 514/456

(57) ABSTRACT

Disclosed is a composition and method for controlling weight gain and/or inducing weight loss in an individual, particularly a human being, in need of such weight loss. The method employs a composition containing one or more isoflavones, such as phytoestrogens, and results in a decrease of appetite, a feeling of fullness upon consumption of lesser amounts of food, and a concomitant loss of weight. The method further avoids the disadvantages associated with stimulant weight-loss drugs, such as caffeine and amphetamine.

### CROSS REFERENCE TO RELATED APPLICATIONS

This application claims the benefit of U.S. Provisional Application No. 60/193,463, filed Mar. 31, 2000, which is incorporated herein by reference.

### FIELD OF THE INVENTION

The present invention relates to a method of treating obesity, inducing weight loss, or preventing weight gain. In particular, the present invention relates to a method comprising administering to a human in need of anti-obesity treatment one or more phytochemicals belonging to the class of phytoestrogens.

### BACKGROUND OF THE INVENTION

Obesity, and especially upper body obesity, is a common and very serious public health problem in the United States and throughout the world. According to recent statistics, more than 25% of the United States population and 27% of the Canadian population are over weight. Kuczmarski, Amer. J. of Clin. Nut. 55: 495S-502S (1992); Reeder et. al., Can. Med. Ass. J., 23:226-233 (1992). Upper body obesity is the strongest risk factor known for type II diabetes mellitus, and is a strong risk factor for cardiovascular disease and cancer as well. Recent estimates for the medical cost of obesity are $150,000,000,000 world wide. The problem has become serious enough that the Surgeon General of the United States has begun an initiative to combat the ever increasing adiposity rampant in American society.

Much of the pathology induced by obesity can be attributed to the strong association with dyslipidemia, hypertension, and insulin resistance. Many studies have demonstrated that reduction in obesity by diet and exercise reduces these risk factors dramatically. Unfortunately, attempts to treat obesity by diet and exercise are generally unsuccessful, with a failure rate reaching 95%. This failure may be due to the fact that obesity condition is strongly associated with genetically inherited factors that contribute to increased appetite, preference for highly caloric foods, reduced physical activity, and increased lipogenic metabolism. This indicates that people inheriting these genetic traits are prone to becoming obese, regardless of their efforts to combat the condition. Therefore, a pharmacological agent that can correct this adiposity condition, and allow physicians to successfully treat obese patients in spite of their genetic inheritance, is needed.

Much attention in recent years has been focused on developing pharmaceutical solutions to the problem of obesity. One pharmaceutical approach to treating obesity is to treat obese patients with drugs that are known to increase metabolism. Stimulants such as caffeine and amphetamine have been found to increase metabolism, and reduce appetite. However, there are drawbacks to the use of stimulants as anti-obesity drugs. Increased cardiac function can stress the cardiopulmonary system of a patient who is being treated for obesity. As the obese patient is already at increased risk for heart attack and stroke, it often undesirable to treat obesity with drugs that put greater stress on potentially overburdened heart muscle and arteries Therefore, there is a need for anti-obesity treatments that do not rely on stimulants to achieve weight loss.

In recent years there has arisen an increased awareness of the beneficial effects of certain types of foods on the human body. For instance, an increase in oat-derived fiber has been linked to a decrease in overall serum cholesterol levels, and in a beneficial shift in the ratio between serum high density lipoprotein (HDL) and low density lipoprotein (LDL). The beneficial effect of oat bran fiber is believed to arise from its ability to adsorb bile salts in the patient's gut. As bile salts are derived from cholesterol in the liver, the body will convert serum cholesterol into bile salts to make up for the oat bran fiber-induced decrease in bile salts in the intestines. Indeed, humans may be genetically programmed to produce otherwise unhealthy levels of serum cholesterol, especially LDL, in the expectation that the diet will be high in foods that share the ability of oat bran fiber to adsorb cholesterol.

Other beneficial properties of food-derived moieties have also been investigated. For instance, Kelly, in U.S. Pat. No. 5,830,887, discloses that some plant-derived compounds, known as phytoestrogens, possess beneficial effects as agents for treating cancer, pre-menstrual syndrome, menopause or hypercholesterolemia. Naturally occurring phytoestrogens, such as isoflavones, are found in plants such as legumes. These include soy, chick peas, lentils, beans (broad, haricot, kidney, lima, navy, etc.), grams (Bengal, horse and green) and clovers. Soy and clover are known to contain the highest levels of isoflavones.

Principal estrogenic and anti-cancer isoflavones are genistein, daidzein, formononetin, and biochanin A. In plants these compounds occur principally in the glycoside form bound to sugars such as glucose. The formulae of these isoflavones are as follows:

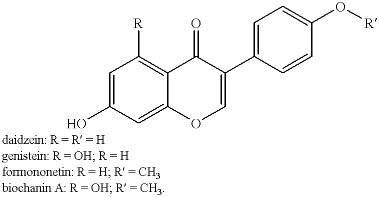

While these compounds are known to possess beneficial biological properties, they have not heretofore been known to be useful for the treatment of obesity.

### SUMMARY OF THE INVENTION

The present invention addresses the need for a method and composition for inducing weight loss that does not possess the detrimental properties associated with prior art methods and compositions employing stimulants. In particular, the present invention provides for a method of inducing weight loss that employs heretofore unidentified properties of isoflavones derived from plants or synthetic sources, with plant-derived isoflavones being more preferred. Suitable methods according to the present invention comprise administering a suitable weight-loss inducing composition comprising one or more isoflavones to a subject in need thereof. The present invention, therefore, provides a method of suppressing weight gain, inducing weight loss, or imparting a feeling of gastric fullness in a subject in need thereof, comprising: administering to the subject an amount of at least one isoflavone sufficient to suppress weight gain, induce weight loss, or impart a feeling of gastric fullness in said subject. The presention invention is also directed to a composition for the treatment of obesity, suppressing weight gain, inducing weight loss, or imparting a feeling of gastric fullness in a subject in need thereof, comprising an isoflavone in an amount effective to treat obesity. The isoflavone may be a phytoestrogen selected from the group consisting of daidzein, genistein, formononetin and biochanin A.

Additional aspects, embodiments and advantages of the present invention will be set forth, in part, in the description that follows, or may be learned from practicing or using the present invention. The objects and advantages may be realized and attained by means of the features and combinations particularly pointed out throughout this written description and the appended claims. It is to be understood that the foregoing general description and the following detailed description are exemplary and explanatory only and are not to be viewed as being restrictive of the invention as claimed.

### DESCRIPTION OF PREFERRED EMBODIMENTS

The present invention relates to a method for inducing weight-loss in an individual, such as a human, by administering to the individual a weight-loss inducing amount of one or more isoflavones, such as the plant-derived isoflavones genistein, daidzein, formononetin and biochanin A, or their natural glycoside form, or their analogues.

Asian peoples have lower body fat compositions than do their counterparts in the West, and in particular in the United States. The present inventor has found that isoflavones, such as the phytoestrogens genistein, daidzein, formononetin and biochanin A, or their natural glycoside form, or their analogues, are surprisingly effective in suppressing appetite and providing individuals who consume them, such as humans, a feeling of fullness. The method according to the present invention takes advantage of this heretofore unknown and surprising property of isoflavones such as genistein, daidzein, formononetin and biochanin A by administering to an individual in need of weight loss a weight loss inducing amount of one or more suitable isoflavone such as genistein, daidzein, formononetin and biochanin A, or their natural glycoside forms, or their analogues, or mixtures thereof.

Individuals treated via the method according to the present invention reported both suppression of appetite and a greater feeling of fullness after eating smaller portions of food than they experienced without treatment according to the present invention.

The method according to the present invention advantageously includes administering to an individual in need of weight loss isoflavones in the form of a composition comprising isoflavones and an excipient, a diluent, a carrier or the like. Such composition may be taken alone or in combination with food. For instance, the composition comprising isoflavone may be mixed with food, or can be consumed directly some time before a meal so as to lower appetite before a meal or between meals. Isoflavones may be isolated from any suitable foodstuffs, that are readily available, have no known toxic components, and are rich sources of isoflavones. Such foodstuffs include red clover and soya.

In certain embodiments of the invention, the ratio of genistein and/or its methylated derivative biochanin A to daidzein and/or its methylated derivative formononetin is between 1:2 to 2:1. In other embodiments of the invention, the ratio of genistein and/or it methylated derivative biochanin A to daidzein and/or its methylated derivative formononetin is not regulated. Other plant components with estrogenic activity including lignans, coumestans and flavones may also be present in the isoflavones administered by the method according to the present invention, but are not critical to the present invention.

A suitable source of isoflavones, which may be administered by the method according to the present invention, is marketed by Novogen under the trade name Promensil/Trinoven. This composition is known to be rich in genistein, daidzein, formononetin, and biochanin A, and to contain relatively lesser quantities of lignans, coumestans and flavones. However, other sources of isoflavones may be used in the method according to the present invention.

Suitable formulations of isoflavones may be found in U.S. Pat. No. 5,830,887 to Kelly, which is incorporated herein by reference.

The method according to the present invention involves administering to an individual in need of weight loss, such as an obese human being, a quantity of isoflavone sufficient to induce weight loss. Exemplary daily dosages of suitable isoflavone are in the range of 5-500 mg/day. A preferred dosage range is 10-200 mg/day, of which a more preferred range is 50-150 mg/day. More preferably, the dosage range is 80-120 mg/day.

While the example below is limited to treatment of human beings, the present invention is not limited to treatment of humans. Other animals, such as domestic dogs and cats, are also treatable by the present method. The term “individual”, unless otherwise qualified, therefore embraces other species of animals, in particular mammals, and even more particularly domesticated mammals.

The method according to the present invention induces weight loss in individuals. An individual in need of weight loss means any individual, including a human or non-human mammal, who is adjudged to be overweight, over-fat or obese according to methods of comparing actual body mass to ideal and/or healthy body mass, which are known to skilled physicians, trainers and dieticians. The method for determining whether the individual is in need of weight loss may be subjective or objective, as exemplified by comparison of actual weight with insurance actuarial tables, or by comparison of the ratio of actual body mass to the actual square of body height with ideal values established by the Surgeon General of the United States.

In a method according to the present invention isoflavones are advantageously administered regularly on a daily basis over a sufficient period for the individual to manifest health-improving weight loss, such as one month to two years. As the method according to the present invention induces weight loss by naturally inhibiting the appetite of the treated individual, and inducing a sense of fullness when the individual consumes smaller food portions, the weight loss will occur gradually and without the negative side effects associated with stimulants such as caffeine and amphetamine. Treatment may therefore be continued as long as is necessary for the individual to attain a healthy weight, and the skilled physician, trainer or dietician can therefore tailor the method to the needs of the individual by varying the length of treatment as necessary.

The method according to the present invention employs a plant-derived isoflavone, such as, for instance a phytoestrogen isoflavone. Phytoestrogens employed in the method according to the present invention may be obtained from a number of different sources. Advantageously the phytoestrogens are extracted from a clover such as red clover or subterranean clover or from soya, which contain high levels of phytoestrogens. However, any source rich in phytoestrogens may be used instead, if desired.

Various different isoflavones have been identified from these sources—they are principally genistein, biochanin A, daidzein, formononetin and glycitein. In plants these compounds occur principally in a glycoside form bound to sugars such as glucose, with smaller amounts present as the aglucone forms. The formulae of the isoflavones are:

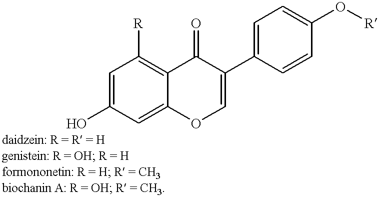

Following ingestion by humans, the glycosidic isoflavones are hydrolyzed to the aglucone form and biochanin A and formononetin are demethylated by bacterial fermentation to genistein and daidzein respectively. A small proportion of these free isoflavones are absorbed directly from the bowel and circulate in the blood. The bulk of the isoflavones, however, remain in the bowel and undergo fermentation to form various metabolites which also are absorbed into the bloodstream. The principal metabolites which have been identified are equol and O-desmethylangolensin.

Any leguminous plants could be used as sources of phytoestrogens (principally isoflavones with lesser amounts of lignans and coumestans). Examples include, without limitation, Indian liquorice (Abrus precatorius); various species of Acacia spp. including, A. aneura, A. cibaria, A. longifolia, and A. oswaldii; ground nut (Apio tuberosa); ground pea (Arachis hypogea); milk vetch (Astragalus edulis); marama bean (Bauhinia esculenta); sword bean (Cajanus cajan indicus); jack bean (Canavalia ensiformis); sword bean (Canavalia gladiata); seaside sword bean (Canavalia rosea); various Cassia spp. including C. floribunda, C. laevigata, and C. occidentalis; carobbean (Ceratonia siliqua); chick pea (Cicer arietinum); yebnut (Cordeauxia edulis); various Crotalaria spp. including C. laburnifolia, and C. pallida; cluster bean (Cyamopsis psoralioides); tallow tree (Detariaum senegalense); sword bean (Entada scandens); balu (Erythrina edulis); soyabean (Clycine max;) inga (Ingaedulis); Polynesian chestnut (Inocarpus fagifer); hyacinth bean (Lablab purpureus); grass pea or Indian vetch (Lathyrus sativus); cyprus vetch (Lathyrus ochrus); lentil (Lens culinaris); jumping bean (Leucaenal eucocephala); various Lupinus spp. including L. albus, L. luteus, L. angustifolium, L. mutabilis, and L. cosentinii; ground bean (Macotylma geocarpa); horse gram (Macrotyloma uniflorum); alfalfa (Medicago sativa); velvet bean (Mucuna pruriens); yam beans (Pachyrhyzuz erosus, P. tuberosus); African locust bean (Parkia clappertoniana); Parkia speciosa; oil bean tree (Pentaclethra macrophylla); various Phaseolus spp. including P. acutifolius, P. vulgaris, P. luntus, P. coccineus, P. adenathus, P. anguiris, P. aureus, P. calcaratus, P. mungo, and P. polystachyus; garden pea (Pisum sativum); djenko bean (Pithecolobium lobatum); mesquite (various Prosopis spp.); goa bean (Psophocarpus scandens, P. tetragonolobus); various Psoralea spp.; Sesbania bispinosa; yam bean (Sphenostylis stenocarpa); tamarind (Tamarindus indica); fenugreek (Trigonella foenum-graecum); vetches (various Vivia spp. including V. sativa, V. atropurpurea, V. ervilia, and V. monantha); broad bean (Vicia faba); black gram (Vigna mungo); various Vigna spp. including V. radiata, V. aconitifolia, V. adanatha, V. angularus, V. tribolata, V. umbelata, and V. unguiculata; earth pea (Voandzeia subterranea), etc.

Suitable sources of phytoestrogens which may be employed in the method according to the present invention are those which (i) are readily available, (ii) are relatively inexpensive, (iii) are readily and economically processed so as to yield the extract, (iv) have a high isoflavone content so as to provide high yields, and (v) have no known toxic components requiring selective removal or inactivation. Of course, the phytoestrogen contemplated herein may be synthetically made by a skilled artisan using readily available starting materials.

Certain clovers, such as red clover (T. pratense) and subterranean clover (T. subterranean) are the particularly suitable sources. On a dry weight basis, these clovers contain the highest amounts of estrogenic isoflavones of all legumes tested to date with levels of 3-5 g % (T. subterranean) and 1-3 g % (T. pratense). In comparison, soya flour has a level of 0.15-0.30 g %, lentils (0.08-0.12 g %), chick peas (0.07-0.13 g %), and garden peas (0.02-0.03 g %). Thus it can be seen that clovers contain approximately at least 10-30 times by weight the isoflavone content of other commonly available, human leguminous foodstuffs meaning that for manufacturing purposes, the yield of isoflavones per unit weight of plant material is many times greater from clover than from other legumes.

The method according to the present invention advantageously employs formulations containing isoflavones, for instance the phytoestrogens discussed above together with a dietary suitable excipient, diluent, carrier, or with a food. The isoflavones may be administered in the form of a pill, tablet, capsule, or similar dosage form. While isoflavones such as the phytoestrogens daidzein, genistein, formononetin and biochanin A are found in naturally occurring plants, they are naturally available only in small concentrations in nature. Therefore, the method according to the present invention advantageously employs isoflavones that have been previously isolated, and thus concentrated, from plants. Another potential source of such isoflavones is by synthetic methods. Advantageously the form and concentration of isoflavones employed in the method according to the present invention is of such that a normal, healthy individual who is treated according to the method according to the present invention will consume less than 100%, preferably less than about 90%, and more preferably less than about 75% of the individual's normal caloric intake.

The formulations employed in the method according to the present invention may be a variety of kinds, such as nutritional supplements, pharmaceutical preparations, vitamin supplements, food additives or foods supplemented with the aforementioned isoflavones, for instance phytoestrogens, which may be included in liquid or solid preparations, including drinks, sterile injectable solutions, tablets, coated tablets, capsules, powders, drops, suspensions, or syrups, ointments, lotions, creams, pastes, gels, or the like. The formulations may be in convenient dosage forms, and may also include other active ingredients, and/or may contain conventional excipients, carriers and diluents. Suitable pharmaceutical-grade carriers are described in Remington's Pharmaceutical Sciences. The method of the present invention may also be practiced by including isoflavones, for instance phytoestrogens, in so called herbal remedies.

The method according to the present invention may be practiced by preparing a composition containing one or more isoflavones, such as one or more of the aforementioned phytoestrogens, in a unit dosage form. The amount of isoflavone in each unit is advantageously a full daily dose, i.e. 5-500, preferably 10-200, more preferably 50-150, even more preferably 80-120 mg per dose, however other unit dosages are contemplated within the present invention. In other preferred embodiments according to the present invention, the total daily dosage is advantageously divided between two, three, four, or up to ten unit doses. In certain embodiments according to the present invention, the total isoflavone dosage is divided into three or four unit doses of about 20 mg/unit dose to about 40 mg/unit dose. In some embodiments according to the present invention, the unit doses are all taken simultaneously, while in other embodiments the unit doses are interspersed throughout the day, so as to maintain a constant level of isoflavone in the subject's body. Subsequently, the dosage or frequency of administration, or both, may be reduced, as a function of weight loss, to a level at which the improved condition is retained when the amount of weight loss has been alleviated to a desired level, treatment should cease. Subjects, may, however, require intermittent treatment on a long-term basis upon any recurrence of undesirable weight gain.

### ILLUSTRATIVE EXAMPLE

The invention is now described with reference to an illustrative example.

### EXAMPLE

Promensil/Trinoven brand isoflavone supplement was obtained from Novogen Research Pty. Ltd. Several overweight individuals were fed 80-120 mg of the isoflavone supplement per day for a period of one month. These individuals experienced weight loss of 10 to 50 lbs. Each of the treated individuals stated that consumption of the tested amount of isoflavone led to reduced appetite and a greater feeling of fullness, and with smaller portions of food, than they experienced in the absence of isoflavone.

### CONCLUSION

The above results show that the method according to the present invention provides for a method of inducing weight loss in individuals, including humans comprising administering to the individuals isoflavones isolated from plants. The method according to the present invention overcomes problems associated with previously known weight loss inducing agents, such as caffeine and amphetamine. By naturally reducing the appetite of individuals, and by giving them a greater sense of fullness, and with smaller amounts of food, than they experienced in the absence of effective amounts of isoflavones, such as genistein, daidzein, formononetin, and biochanin, the method according to the present invention induces healthy weight loss in individuals without such negative side effects as nervousness, and increased risk for hypertension, heart attack, and stroke that are associated with methods employing stimulants such as caffeine and amphetamine. Thus, the present invention represents an improved method for treating individuals, such as humans, who are at increased health risk due to excessive body mass.

While the present invention has been described in connection with an illustrative example, it is to be understood that the invention is not limited to this example, but, on the contrary, is intended to cover various modifications and equivalent arrangements included within the scope of the appended claims.

What is claimed is:

1. A method of suppressing weight gain, inducing weight loss, or imparting a feeling of gastric fullness in a subject in need thereof, comprising:
administering to the subject an amount of at least one isoflavone sufficient to suppress weight gain, induce weight loss, or impart a feeling of gastric fullness in said subject.

2. The method of claim 1, wherein the subject is a human.

3. The method of claim 1, wherein at least one isoflavone is selected from the group consisting of genistein, daidzein, formononetin, and biochanin A.

4. The method of claim 1, wherein the amount of isoflavone administered to the subject is in the range of about 5 to about 500 mg/day.

5. The method of claim 4, wherein the amount of isoflavone administered to the subject is in the range of about 10 to about 200 mg/day.

6. The method of claim 5, wherein the amount of isoflavone administered to the subject is in the range of about 50 to about 150 mg/day.

7. The method of claim 6, wherein the amount of isoflavone administered to the subject is in the range of about 80 to about 120 mg/day.

8. The method of claim 7, wherein the amount of isoflavone administered to the subject is about 100 mg/day.

9. A composition for the treatment of obesity, comprising an isoflavone in an amount effective to treat obesity.

10. A composition according to claim 9, wherein at least the isoflavone is selected from the group consisting of genistein, daidzein, formononetin, and biochanin A.

11. A composition according to claim 9 in unit dosage form.

12. A composition according to claim 11, wherein the amount of isoflavone in the unit dose is such that the effective amount is contained in from one to ten unit doses.

13. A composition according to claim 12, wherein the effective amount is from about 5 to about 500 mg/day.
